(19) United States
(12) Patent Application Publication
(10) Pub. No.: US 2023/0330196 A1
(43) Pub. Date: Oct. 19, 2023

(54) IMMUNOSUPPRESSIVE AGENTS

(71) Applicant: The Trustees of Columbia University in the City of New York, New York, NY (US)

(72) Inventors: Suzanne Lentzsch, Bronx, NY (US); Shirong Li, Paramus, NJ (US); Jing Fu, Paramus, NJ (US); Markus Y. Mapara, New York, NY (US); Hui-Hui Ma, Tenafly, NJ (US)

(21) Appl. No.: 18/310,702

(22) Filed: May 2, 2023

Related U.S. Application Data
(63) Continuation-in-part of application No. PCT/US2021/057804, filed on Nov. 3, 2021.
(60) Provisional application No. 63/109,374, filed on Nov. 4, 2020.

(51) Int. Cl. A61K 38/48; A61P 37/06; C12N 9/64

(52) U.S. Cl. CPC A61K 38/4886; A61P 37/06; C12N 9/6424; C12Y 304/21

(57) ABSTRACT

Immunosuppressive peptide fragments of MMP-13, pharmaceutical compositions comprising said peptides, and methods of inhibiting T cell proliferation.

### CROSS-REFERENCE TO RELATED APPLICATIONS

This application is a continuation-in-part of International Patent Application No. PCT/US2021/57804, filed on Nov. 3, 2021, which claims priority from U.S. provisional patent application 63/109,374, filed Nov. 4, 2020, which application is incorporated herein in its entirety.

### GOVERNMENT RIGHTS

This invention was made with United States government support under Contacts CA175313 and CA252756, awarded by the National Institutes of Health. Thus, the United States government may have certain rights to the invention described and claimed herein.

### REFERENCE TO A SEQUENCE LISTING SUBMITTED BY EFS-WEB

The contents of the Sequence Listing XML file, named 8441-0030WOUS.xml, created on Mar. 13, 2023, which is 27,058 bytes in size, and which is electronically submitted with this application, is hereby incorporated herein by reference in its entirety.

### FIELD OF THE INVENTION

The present disclosure relates to methods of inhibiting T cell proliferation, T cell proliferation inhibiting peptide fragments of the N-terminal domain of matrix-metalloproteinase 13 (“MMP-13”), and pharmaceutical compositions comprising the N-terminal domain of MMP-13 or said peptides.

### BACKGROUND OF THE INVENTION

The following discussion is provided merely to aid the reader in understanding the disclosure and is not admitted to describe or constitute prior art thereto.

Matrix metalloproteinases (MMPs) have been recognized for their role in degradation of extracellular matrix (ECM) and collagen remodeling and have also been shown to play a crucial role in inflammation, tumor cell invasion, and adaptive and innate immunity.

Multiple myeloma (MM) bone disease is characterized by the development of osteolytic bone lesions due to the over-activation of osteoclast and inhibition of osteoblast cells. MM cells secret pro-osteoclastogenic factors which lead to osteoclast (OCL) activation. Matrix metalloproteinase 13 (MMP-13) is a critical osteoclastogenic factor which is highly secreted by MM cells. MMP-13 induces osteoclast fusion and bone-resorption by triggering the ERK1/2-dependent up-regulation of the cell fusogen, DC-STAMP. Li et al., Arthritis Research and Therapy (2017) 19: 248; Fu et al., J. Clin. Invest. 2016, 126(5): 1759-1722.

Negative checkpoint regulators temper the T cell immune response to self-antigens and limit the development of auto-immunity. One important such checkpoint regulator is the V-type immunoglobulin domain-containing suppressor of T-cell activation (VISTA) (also known as programmed death-1 homolog; “PD-1H”), which is broadly expressed on cells of the myeloid and lymphoid lineages. Since VISTA is often implicated as a poor prognostic indication in cancer, it is a promising target for improving cancer immunotherapy. El Tanbouly et al., Chem. and Exptl. Immunology 200: 120-130 O(2020)

Matrix metalloprotein 13 (MMP-13) has the following sequence:

(SEQ ID NO: 1)
MHPGVLAAFLFLSWTHCRALPLPSGGDEDDLSEEDLQFAERYLRSYYH
PTNLAGILKENAASSMTERLREMQSFFGLEVTGKLDDNTLDVMKKPRC
GVPDVGEYNVFPRTLKWSKMNLTYRIVNYTPDMTHSEVEKAFKKAFKV
WSDVTPLNFTRLHDGIADIMISFGIKEHGDFYPFDGPSGLLAHAFPPG
PNYGGDAHFDDDETWTSSSKGYNLFLVAAHEFGHSLGLDHSKDPGALM
FPIYTYTGKSHFMLPDDDVQGIQSLYGPGDEDPNPKHPKTPDKCDPSL
SLDAITSLRGETMIFKDRFFWRLHPQQVDAELFLTKSFWPELPNRIDA
AYEHPSHDLIFIFRGRKFWALNGYDILEGYPKKISELGLPKEVKKISA
AVHFEDTGKTLLFSGNQVWRYDDTNHIMDKDYPRLIEEDFPGIGDKVD
AVYEKNGYIYFFNGPIQFEYSIWSNRIVRVMPANSILWC.

MMP-13 comprises an N-terminal domain (amino acids 20-267):

(SEQ ID NO: 2)
LPLPSGGDEDDLSEEDLQFAERYLRSYYHPTNLAGILKENAASSMTER
LREMQSFFGLEVTGKLDDNTLDVMKKPRCGVPDVGEYNVFPRTLKWSK
MNLTYRIVNYTPDMTHSEVEKAFKKAFKVWSDVTPLNFTRLHDGIADI
MISFGIKEHGDFYPFDGPSGLLAHAFPPGPNYGGDAHFDDDETWTSSS
KGYNLFLVAAHEFGHSLGLDHSKDPGALMFPIYTYTGKSHFMLPDDDV
QGIQSLYG,

a hinge domain (amino acids 268-280), and a hemopexin domain (amino acids 281-471). The N-terminal domain comprises a signal region (amino acids 1-19), a pro-peptide region (amino acids 20-103) and a catalytic domain (amino acids 104-267).

### SUMMARY OF THE INVENTION

The inventors have discovered that MMP-13 and peptide fragments thereof are able to suppress T cell proliferation in vitro and in vivo. This activity is independent of the catalytic activity of MMP-13. Described herein are methods of inhibiting T cell proliferation, T cell proliferation inhibiting peptide fragments of the N-terminal domain of MMP-13, and pharmaceutical compositions comprising the N-terminal domain of MMP-13 or said immunosuppressive peptides.

It has been discovered that checkpoint inhibitor PD-1H is highly expressed in pre- and mature osteoclasts (OCL) and that it is the critical receptor for MMP-13 in OCL activation, thereby mediating MMP-13-induced OCL fusion, activation and bone resorption. Hence, MMP-13 and immunosuppressive peptide fragments thereof play a role in the regulation of T-cell activity in MM. As such, targeting MMP-13 and PD-1H interactions using immunosuppressive peptide fragments of MMP-13 is a novel therapeutic strategy to treat MM bone disease and modulate the MM immune environment.

In one aspect, the invention provides a method of inhibiting T cell proliferation in a subject in need of such inhibition that comprises administering to said subject a T cell proliferation inhibiting amount of the N-terminal domain of MMP-13 (SEQ ID NO: 2) or of a peptide comprising at least six contiguous amino acid residues of the N-terminal domain of MMP-13.

In another embodiment, the present invention provides immunosuppressive peptides comprising at least six contiguous amino acid residues of the N-terminal domain of MMP-13. The peptides may be selected from any part of the N-terminal domain (SEQ ID NO.2), but preferably they are selected from the pro-peptide region (amino acids 20-103): AERYLRSYYHPTNLAGILKENAASSMTERLREMQSFFGLEVTGKLDDNTLDV MKKPRCGVPDVGEYNVFPRTLKWSKMNLTYRIVNYTPDMTHSEVEKAFKKA (SEQ. ID NO: 3) or from amino acids 245-256, having the sequence TYTGKSHFMLPD (SEQ ID NO: 4). Although the subject immunosuppressive peptides may be of any length greater than six amino acid residues, they are preferably between six and about 25 or between six and about 20 or between six and about 15 contiguous amino acid residues of the N-terminal domain of MMP-13.

Included among the subject immunosuppressive peptides are (for example) peptides comprising the sequences: TYTGKS (SEQ ID NO: 5), HFMLPD (SEQ ID NO: 6), TYTGKSHFM (SEQ ID NO: 7), AERYLRS (SEQ ID NO:8), YHPTNLAGIL (SEQ ID NO:9), KENAASSM (SEQ ID NO:10), MTERLREMQ (SEQ ID NO:11), FGLEVTGK (SEQ ID NO:12), LDDNTLDVMKK (SEQ ID NO:13), PRCGVPDVG (SEQ ID NO:14), EYNVFPR (SEQ ID NO:15), KWSKMNLTYR (SEQ ID NO:16), IVNYTPDMT (SEQ ID NO:17), and HSEVEKAFKKA (SEQ ID NO:18).

The subject peptides may comprise terminal additions on the amino and/or carboxyl termini provided that these additions do not materially interfere with the ability of the peptides to inhibit T cell proliferation. These terminal additions may be selected from amino acid residues, and (in the case of the carboxyl terminal group) an amide group unsubstituted or mono- or di-substituted with a C₁₋₄ alkyl group. N-terminal acylation or C-terminal amidation are also possible terminal additions. See: Thomas, A. —Towards a Functional Understanding of Protein N-terminal Acylation, PLOS Biol. 2011, 9(5); and Kim K. and Seong, B. L. —Peptide Amidation: Production of Peptide Hormones in vivo and in vitro. Biotechnol. Eng. 2001, 6(4), 244-251.

In another aspect, the present invention provides pharmaceutical compositions comprising the N-terminal domain of MMP-13 or a peptide comprising at least six contiguous amino acid residues of said N-terminal domain of MMP-13.

### BRIEF DESCRIPTION OF THE DRAWINGS

FIG. 1 shows recombinant MMP-13 suppresses proliferation of allo-reactive T cells independent of catalytic function.

FIG. 2 shows lack of MMP13 in recipients promotes in vivo expansion of T cells.

FIG. 3 shows lack of MMP13 mitigates upregulation of PD-L1 in response to LPS.

FIG. 4 shows overexpression of MMP13 in 5TGM myeloma cells promotes expression of PD-L1 in antigen presenting cells of myeloma microenvironment.

FIG. 5 shows the results of binding experiments between MMP-13 and PD-1H.

### DETAILED DESCRIPTION OF THE INVENTION

In accordance with the present disclosure, a method of inhibiting T cell proliferation in a subject in need of such inhibition is provided which comprises administering to the subject a T cell proliferation inhibiting amount of the N-terminal domain of MMP-13 or of a T cell proliferation inhibiting peptide comprising at least six contiguous amino acid residues of said N-terminal domain of MMP-13.

In another aspect of the present invention, T cell proliferation inhibiting peptides are provided that comprise at least six contiguous amino acid residues of the N-terminal domain of MMP-13. Preferably said peptides comprise between six and about 25 contiguous amino acid residues of the N-terminal domain of MMP-13. More preferably, said peptides comprise between six and about 20 contiguous amino acid residues of the N-terminal domain of MMP-13. Still more preferably, said peptides comprise between six and about 15 contiguous amino acid residues of the N-terminal domain of MMP-13.

The peptides may be selected from any part of the N-terminal domain (SEQ ID NO.2), but preferably they are selected from the pro-peptide region (amino acids 20-103): AERYLRSYYHPTNLAGILKENAASSMTERLREMQSFFGLEVTGKLDDNTLDV MKKPRCGVPDVGEYNVFPRTLKWSKMNLTYRIVNYTPDMTHSEVEKAFKKA (SEQ. ID NO: 3) or from amino acids 245-256, having the sequence TYTGKSHFMLPD (SEQ ID NO: 4).

Thus, the subject peptides may comprise from 6 to 25, from 6 to 20 or from 6 to 15 continuous amino acid residues starting at amino acid 20 of MMP-13 or at any other amino acid through amino acid 261 of MMP-13. Typical examples of the subject peptides include TYTGKSHFMLPD (SEQ ID NO: 4), TYTGKS (SEQ ID NO: 5), HFMLPD (SEQ ID NO: 6), TYTGKSHFM (SEQ ID NO: 7), AERYLRS (SEQ ID NO:8), YHPTNLAGIL (SEQ ID NO:9), KENAASSM (SEQ ID NO:10), MTERLREMQ (SEQ ID NO:11), FGLEVTGK (SEQ ID NO:12), LDDNTLDVMKK (SEQ ID NO:13), PRCGVPDVG (SEQ ID NO:14), EYNVFPR (SEQ ID NO:15), KWSKMNLTYR (SEQ ID NO:16), IVNYTPDMT (SEQ ID NO:17), and HSEVEKAFKKA (SEQ ID NO: 18).

The subject peptides may comprise terminal additions on the amino and/or carboxyl termini provided that these additions do not materially interfere with the ability of the peptides to inhibit T cell proliferation. These terminal additions may be selected from amino acid residues, and (in the case of the carboxyl terminal group) an amide group unsubstituted or mono- or di-substituted with a C₁₋₄ alkyl group. As described above, N-terminal acylation or C-terminal amidation are also possible terminal additions. See: Thomas, A. —Towards a Functional Understanding of Protein N-terminal Acylation, PLOS Biol. 2011, 9(5); and Kim K. and Seong, B. L. —Peptide Amidation: Production of Peptide Hormones in vivo and in vitro. Biotechnol. Eng. 2001, 6(4), 244-251.

The subject peptides may also comprise or be attached to molecules to enhance their absorption of to cause their sustained release, such as polyethylene glycol (PEG) or poly-lactic-co-glycolic acid (PLGA), as are known in the art. See: F. M. Veronese, Peptide and Protein PEGylation: a review of problems and solutions. Biomaterials 2001, 22, 405-417.

Also provided are pharmaceutical compositions comprising the N-terminal domain of MMP-13 or a T cell proliferation inhibiting peptide comprising at least six contiguous amino acid residues of said N-terminal domain of MMP-13 in admixture with a pharmaceutically acceptable carrier as described below.

### Abbreviations and Definitions

As used herein, certain terms may have the following defined meanings.

As used herein, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the subject peptides and methods include the recited elements and steps, without excluding others. “Consisting essentially of” when used to define the subject peptides or methods, shall mean excluding other elements or steps of any essential significance to the subject peptides or methods. “Consisting of” shall mean excluding more than trace elements of other materials for the subject peptides or methods. Embodiments defined by each of these transition terms are within the scope of this disclosure. Accordingly, it is intended that the subject peptides can include additional elements (comprising) or alternatively include additional elements of no significance (consisting essentially of) or alternatively, only the stated subject peptides (consisting of).

Thus, the description of the subject peptides as “comprising” from 6 to 25 or 6 to 20 or 6 to 15 contiguous amino acid residues of the N terminal region of MMP-13 means the subject peptides may include additional chemical moieties on the amino and/or carboxy termini of the 6 to 25, 6 to 20, or 6 to 15 contiguous amino acid residues of the N terminal region of MMP-13, as described above.

These additional chemical moieties may be additional contiguous amino acid residues of the N terminal region of MMP-13. For example, a subject peptide described as “comprising YHPTNLAGIL (SEQ ID NO:9)” may comprise additional contiguous amino acid residues from the N terminal region of MMP-13 on the amino or carboxy termini, such as (for example) RSYYYHPTNLAGIL (SEQ ID NO:19), YHPTNLAGILKENA (SEQ ID NO:20), or RSYYHPTNLAGILKEN (SEQ ID NO:21). These peptides SEQ ID NO:19, SEQ ID NO: 20, AND SEQ ID NO: 21, as well as others in which SEQ ID NO: 9 has contiguous amino acid moieties from the N terminal region of MMP-13 on its termini, are considered to “comprise” YHPTNLAGIL (SEQ ID NO:9).

As used herein, “about” means plus or minus 10%.

As used herein, “optional” or “optionally” means that the subsequently described event or circumstance may or may not occur, and that the description includes instances where said event or circumstance occurs and instances where it does not.

As used herein, the terms “individual”, “patient”, or “subject” can be an individual organism, a vertebrate, a mammal (e.g., a bovine, a canine, a feline, or an equine), or a human. In a preferred embodiment, the individual, patient, or subject is a human.

As used herein, the term “contiguous” when applied to the amino acid residues of the N terminal region of MMP-13 means that the amino acid residues occur in the same order as in MMP-13 and without any intervening chemical entities.

Amino acids are represented by the IUPAC abbreviations, as follows: Alanine (Ala; A), Arginine (Arg; R), Asparagine (Asn; N), Aspartic acid (Asp; D), Cysteine (Cys; C), Glutamine (Gln; Q), Glutamic acid (Glu; E), Glycine (Gly; G), Histidine (His; H), Isoleucine (Ile; I), Leucine (Leu; L), Lysine (Lys; K), Methionine (Met; M), Phenylalanine (Phe; F), Proline (Pro; P), Serine (Ser; S), Threonine (Thr; T), Tryptophan (Trp; W), Tyrosine (Tyr; Y), Valine (Val; V). Similarly for nucleotides: Adenine (A), Cytosine (C), Guanine (G), Thymine (T), Uracil (U), Adenine or Guanine (R), Cytosine or Thymine (Y), Guanine or Cytosine (S), Adenine or Thymine (W), Guanine or Thymine (K), Adenine or Cytosine (M), Cytosine or Guanine or Thymine (B), Adenine or Guanine or Thymine (D), Adenine or Cytosine or Thymine (H), Adenine or Cytosine or Guanine (V), and any base (N).

### Preparation of Peptides

The disclosed peptides may be prepared by methods well-known in the art, in particular by so-called “Merrifield synthesis”, although they may also be prepared by the techniques of molecular biology. Using recombinant synthesis techniques, an appropriate nucleic acid sequence coding for the desired peptide is readily derived from the peptide sequence and the nucleic acid sequence then spliced into an appropriate vector for expression of the peptide. See, for example, “Merrifield Solid Phase Peptide Synthesis” in Wang—“Comprehensive Organic Name Reactions and Reagents”, September 2010 (Wiley).

### Pharmaceutical Formulations

Pharmaceutical compositions suitable for delivery of the N-terminal domain of MMP-13 or the subject peptides comprise the N-terminal domain of MMP-13 or the desired peptide and a pharmaceutically acceptable carrier or diluent.

The pharmaceutical composition may be formulated for intravenous, subcutaneous, intraperitoneal, intramuscular, oral, nasal, pulmonary, ocular, vaginal, or rectal administration. In some embodiments, the subject peptides are formulated for intravenous, subcutaneous, intraperitoneal, or intramuscular administration, such as in a solution, suspension, emulsion, liposome formulation, etc. The pharmaceutical composition can be formulated to be an immediate-release composition, sustained-release composition, delayed-release composition, etc., using techniques known in the art.

Pharmacologically acceptable carriers for various dosage forms are known in the art. For example, excipients, lubricants, binders, and disintegrants for solid preparations are known; solvents, solubilizing agents, suspending agents, isotonicity agents, buffers, and soothing agents for liquid preparations are known. In some embodiments, the pharmaceutical subject peptides include one or more additional components, such as one or more preservatives, antioxidants, stabilizing agents and the like.

Additionally, the disclosed pharmaceutical subject peptides can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In some embodiment, it will be preferable to include isotonic agents, for example, sugars, polyalcohols such as mannitol, sorbitol, or sodium chloride in the composition. Prolonged absorption of the injectable subject peptides can be brought about by including in the composition an agent that delays absorption, for example, monostearate salts and gelatin.

Sterile injectable solutions can be prepared by incorporating the peptide in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

### Experimental Procedure

To screen for MMP-13 cellular binding proteins/receptors, we performed an MMP-13 pull-down assay wherein recombinant MMP-13-Hiss was incubated with mouse mononuclear bone marrow cells (BMCs) lysates and Ni-NTA magnetic beads were used to pull-down MMP-13-associated proteins. Following mass spectral analysis, programmed death-1 homolog (PD-1H) was identified as a major MMP-13-binding protein. PD-1H, also known as V-domain Ig suppressor of T cell activation (VISTA), is a critical negative checkpoint regulator expressed on myeloid cells and involved in immune responses.

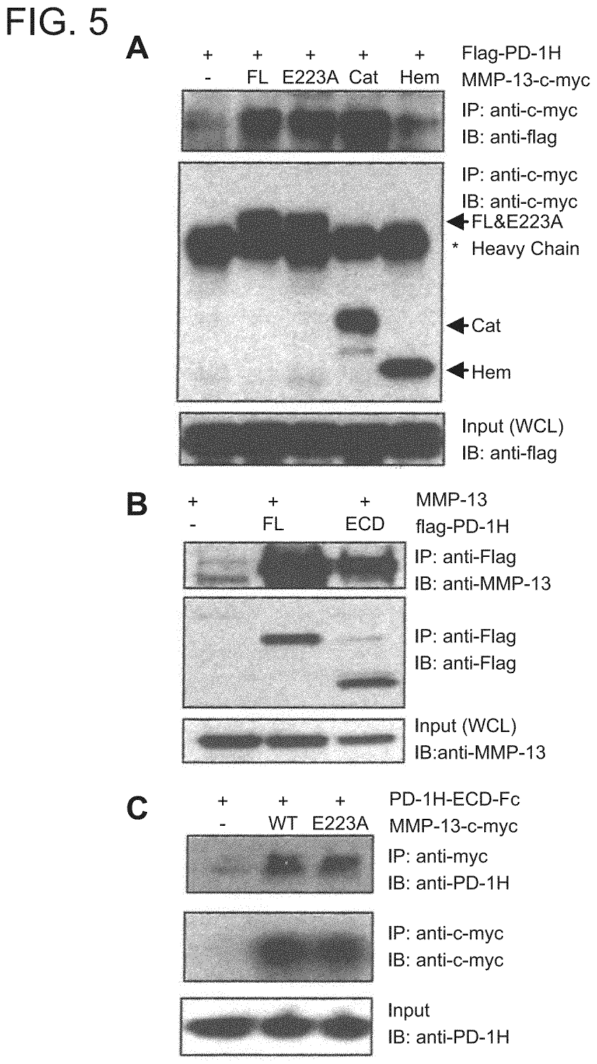

To identify key binding domains between PD-1H and MMP-13, a series of truncated deletion mutants was tested. The MMP-13 zymogen has a pro-peptide domain (aa 20-103) that maintains latency, a catalytic domain (aa 103-268) containing the catalytic zinc-binding motif, and a hinge linker region (aa 269-280) that connects the catalytic domain region with the C-terminal hemopexin domain (aa 281-471). MMP-13 enzymatic activity is not required for binding to PD-1H as demonstrated by the similar binding activity of PD-1H to pro-MMP-13 WT full length (FL) and a catalytically-inactive E223A point mutant (FIG. 5A). By contrast, while the MMP-13 pro-catalytic domain alone (Cat, i.e., aa 1-267) displayed a binding affinity to PD-1H that was similar to full-length MMP-13, a truncation mutant containing the hemopexin domain (Hem, i.e., Δaa 37-267) retained only a fraction of the wild-type binding activity.

PD-1H is a transmembrane protein with N-terminal extracellular domain (aa 30-190), a single-pass transmembrane domain (aa 193-213) and a C-terminal intracellular domain (aa 214-310). In co-immunoprecipitation (co-IP) assays only the extracellular domain (ECD) of PD-1H bound to MMP-13 when co-expressed in HEK 293 cells, although binding activity was somewhat weaker than that of full-length (FL) PD-1H (FIG. 5B). To confirm the direct binding between PD-1H ECD and MMP-13, we incubated a purified MMP-13-myc recombinant protein with a recombinant PD-1H ECD-Fc protein, and performed a pull-down assay using c-myc-tag magnetic beads. Under these conditions, PD-1H ECD-Fc directly bound to MMP-13. Consistent with the lack of any requirement for MMP-13 proteolytic activity, MMP-13 WT and MMP-13 E223A displayed similar binding to PD-1H ECD Fc (FIG. 5C).

Western blot and immunohistochemistry revealed that PD-1H was highly expressed in mononuclear BMCs, pre-OCL and mature OCL. ShRNA-mediated knockdown of PD-1H in mouse mononuclear BMCs blocked the ability of MMP-13 to induce osteoclast fusion and activation. These results were further confirmed by using BMCs from Pd-1h^{-/-} mice and WT littermates for in vitro osteoclast differentiation. While MMP-13 induced WT OCL fusion and activation, these effects were completely blocked in Pd-1h^{-/-} OCLs in tandem with a loss in MMP-13 induced ERK1/2 phosphorylation, NFATc1 and DC-STAMP upregulation.

We have discovered a previously unknown role of MMP-13 in regulating GVHD. To address the function of MMP-13 in GVHD we first assessed the effect of MMP-13 on allo-responses in vitro. Using fully Major Histocompatibility Complex (MHC)-mismatched standard mixed lymphocyte reaction we demonstrated that antigen presenting cells (APC) from B6.MMP-13^{-/-}(H2^{b}) mice led to significantly enhanced antigen-driven activation and proliferation of Carboxyfluorescein succinimidyl ester (CFSE)-labeled Balb/c responder splenocytes. Thus, MMP-13 deficiency in either splenocytes or bone marrow-derived dendritic cells used as stimulators resulted in enhanced proliferation, activation and IFN-g production in the allo-reactive lymphocyte responders. Similarly, exogenous MMP13 reduced proliferation of responder T cells as determined tested by CFSE dilution (CFSE^{lo} of CD4⁺T cells from 62.3% decreased to 40.6%, CFSE^{lo} of CD8⁺T cells from 74.1% down to 47.9%).

We next assessed the impact of MMP-13 in vivo using fully MHC-mismatched rodent acute GVHD models. To study the role of host-derived MMP-13 we induced GVHD in B6.MMP-13^{-/-} or B6. WT recipient mice following lethal TBI (1075 rad) using splenic T cells from Balb/c donors. We observed significantly accelerated GVHD-related mortality (Median Survival Time 7 vs. 47 days post-transplant, p<0.05) in MMP-13-deficient recipients. Most importantly, donor T cells expanded more vigorously in the secondary lymphoid organs (Spleen and mesenteric lymph nodes) of MMP13^{-/-} compared to wildtype recipient mice (e.g. spleen: absolute donor CD4⁺Tcells 1.5×10⁴±7.3×10³ (WT) vs. 5.83×10⁴±1.65×10⁴[MMP-13^{-/-}] and CD8⁺5.5×10⁴±3.8×10⁴ (WT) vs 3.4×10⁵±1.4×10⁵[MMP-13^{-/-}], p<0.01). Enhanced donor lymphocyte expansion was further confirmed by bioluminescence imaging.

To further delineate the underlying mechanisms, we analyzed the effects of MMP-13 deficiency and exogenous MMP-13 on maturation of mouse bone marrow derived-dendritic cells (BMDC) and macrophages in vitro. We noted decreased expression of inhibitory molecules PD-L1 and PD-1H on GM-CSF/LPS cultured BMDC. Similarly, bone marrow-derived MMP-13^{-/-} macrophages also showed reduced PD-L1 and PD-1H expression upon LPS stimulation when compared to their WT counterparts. In summary, the recipient myeloid cell-derived MMP-13 mitigates GVHD and limits donor T cell expansion.

Materials and Methods

Mixed lymphocyte reaction (MLR): 5×10e5 splenocytes (SPCs) from Balb/c mice were labeled with Carboxyfluorescein succinimidyl ester (CFSE), and then cocultured with 5×10e5 irradiated MMP13^{-/-} or WT SPCs, or 1×10e5 irradiated MMP13^{-/-} or WT LPS matured bone marrow derived dendritic cells (LPS-BMDC) for 4 days. The proliferation and activation of T cells was assessed with flow cytometry. For the exogenous MMP13 experiments, CFSE labeled Balb/c SPCs were stimulated with 5×10e5 irradiated PD-1H or WT SPCs in the presence of 200 ng/ml rhMMP13 protein (R&D) for 4 days.

LPS-BM derived DC(BMDC) preparation: Bone marrow cells were flushed and then cultured in medium containing murine GM-CSF (20 ng/ml, Peprotech). On day 6-7, nonadherent cells were harvested, and CD11c⁺ DC were selected with anti-CD11C⁺ beads (purity>90%, Miltenyi Biotec) and matured with LPS (100 ng/ml). After 24 hr stimulation with LPS, nonadherent cells were harvested and irradiated at 3000 cGy.

BMDM (BM derived macrophages) preparation: Bone marrow cells were flushed and then cultured in medium containing murine M-CSF (20 ng/ml, Peprotech). On day 5-6, adherent cells were digested with cell dissociation solution (non-enzymatic, Thermofisher), and then stimulated with LPS (100 ng/ml). After 24 hr stimulation with LPS, adherent cells were harvested for flow cytometric analysis.

BMT and Induction of GVHD: GVHD was induced in the fully MHC-mismatched strain combination BALB/c to B6 or B6.MMP13^{-/-} combination. Recipients were lethally irradiated (1075 cGy) and reconstituted with a single intravenous inoculum of 5×10e6 T cell depleted (TCD) bone marrow cells (BMCs) and 3-7×10⁶ Pan T from BALB/C mice. TCD was performed using CD90.2 microbeads (Miltenyi Biotec), Pan T cells were purified from splenocytes by using PanT isolation kit (Miltenyi Biotec).

Establishment of mouse myeloma model: For the overexpression of MMP-13 in 5TGM1 cells, full-length MMP-13 was cloned into the retroviral vector pLHCX (Clontech). 5TGM1-luc cells were infected by the retrovirus pLHCX-MMP-13 (MMP-13 OE) or pLHCX empty vector (EV), and positively transduced cells were selected by hygromycin B (0.5 mg/ml). MMP-13 OE was confirmed by western blotting using both the whole cell lysates and the cell-conditioned medium. Following, 2×10⁶ 5TGM1-luc EV or MMP-13 OE cells were intratibially injected into C57BL/KaLwRij mice (male, 4-5 wks old). Tumor progression was monitored by weekly bioluminescence imaging (BLI) until week 3. On week 3 following 5TGM1 injection, mice were euthanized, and spleen and tibiae were harvested for flow cytometry analysis.

Flow cytometric analysis: Single-cell suspensions were filtered through nylon mesh and stained with the following antibodies: CD3, CD4, CD8, CD25, IFN-g, PD-L1, CD11c, B220, CD11b, F4/80, and then analyzed by FCM using Fortessa.

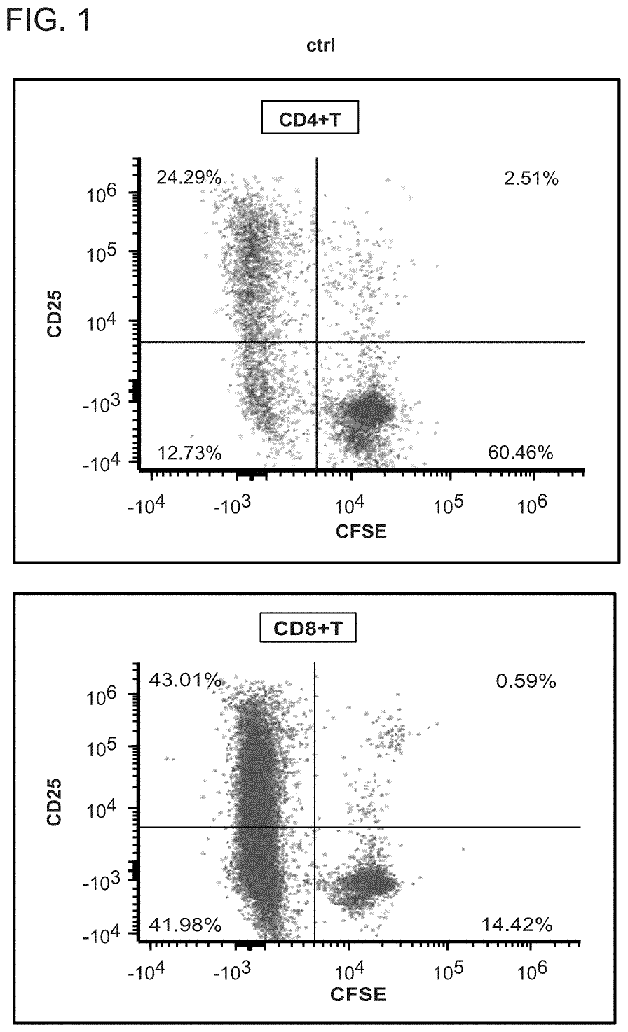

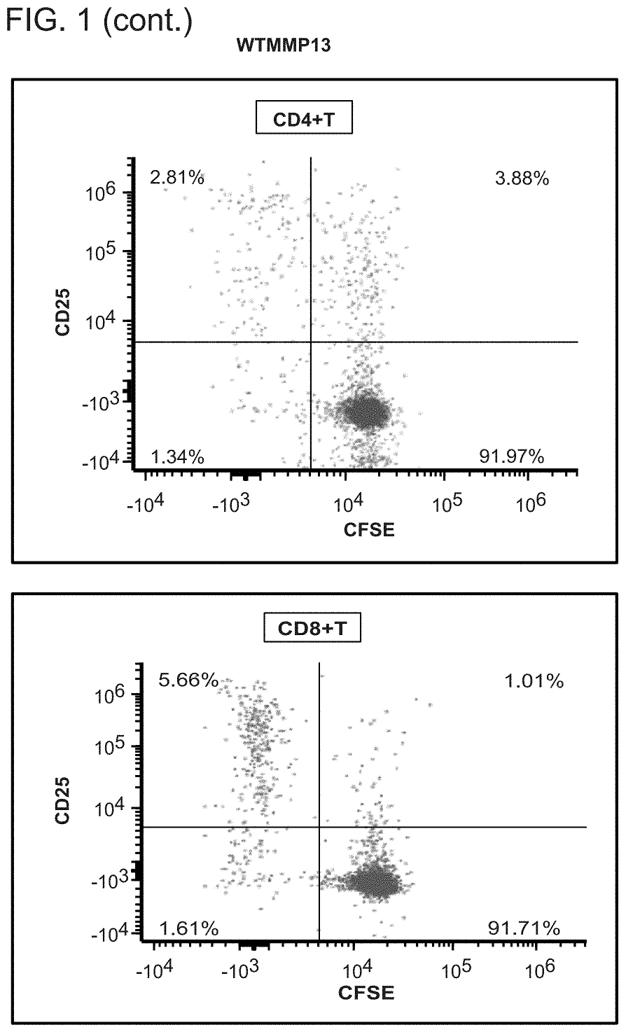

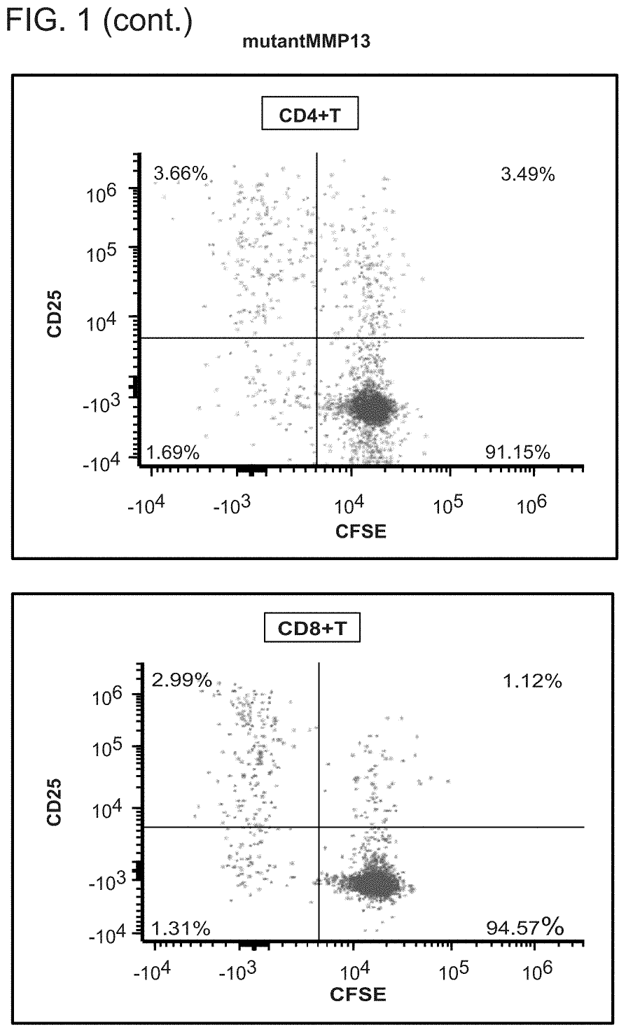

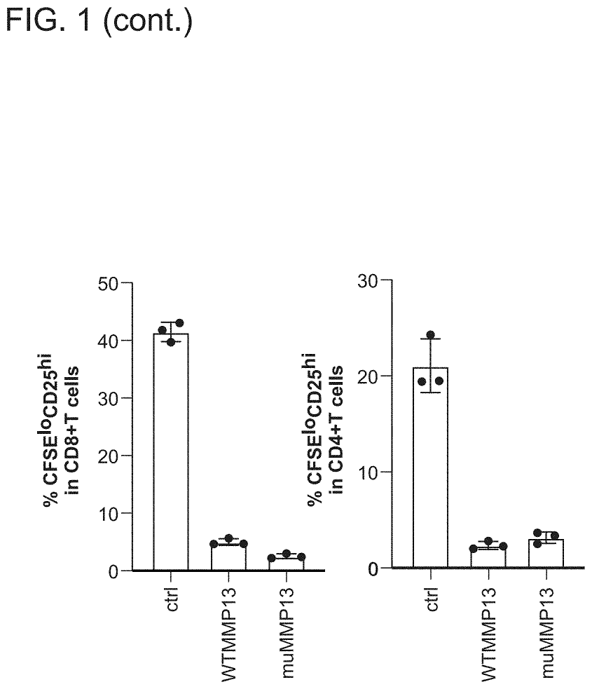

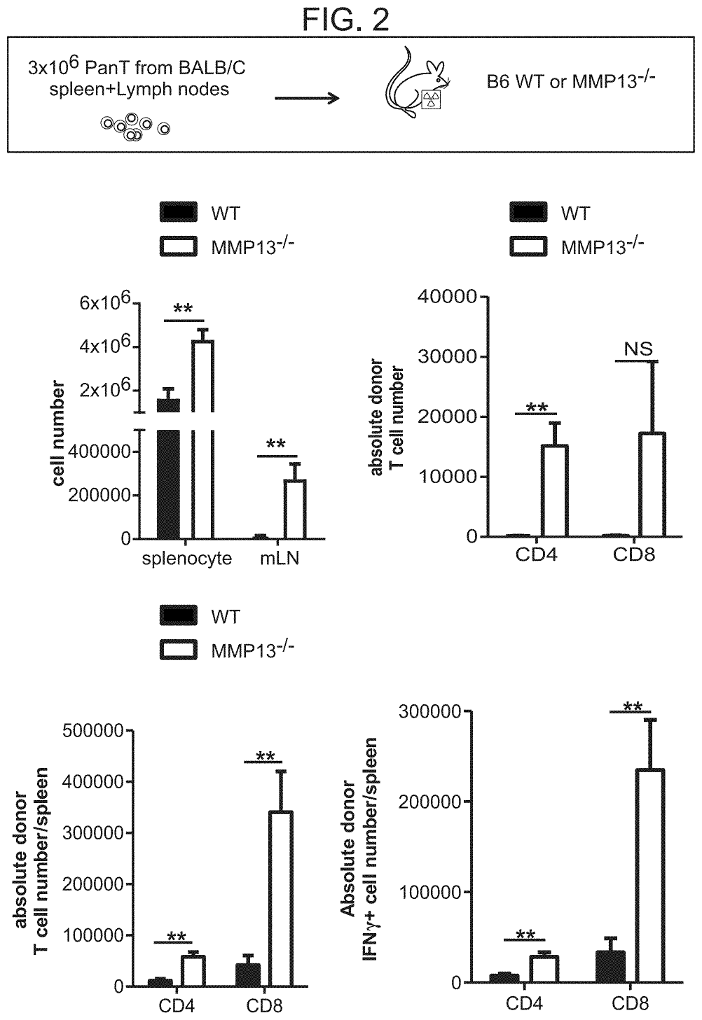

SPCs from B6 mice were seeded at 5×10e5/well, cocultured with irradiated BALB/c SPCs (5×10e5/well) in the presence of recombinant human MMP13 (WT-MMP13) or mutant MMP13 (E223A, muMMP13) proteins for 5 days. Exogenous MMP13 reduced proliferation of responder T cells as determined by CFSE dilution and CD25 expression (CFSE^{lo}CD25+ of CD4+T cells from 21.1% decreased to 2.4% (WT-MMP13) and 3.2% (mu-MMP13), CFSE^{lo}CD25+ of CD8+ T cells from 41.5% down to 5.0% (wt-mmp13) and 2.5% (mu-MMP13). It suggested that MMP13 is a negative regulator of T cells, and exogenous MMP13 suppresses proliferation of allo-reactive T cells independent of catalytic function. (FIGS. 1 and 2)

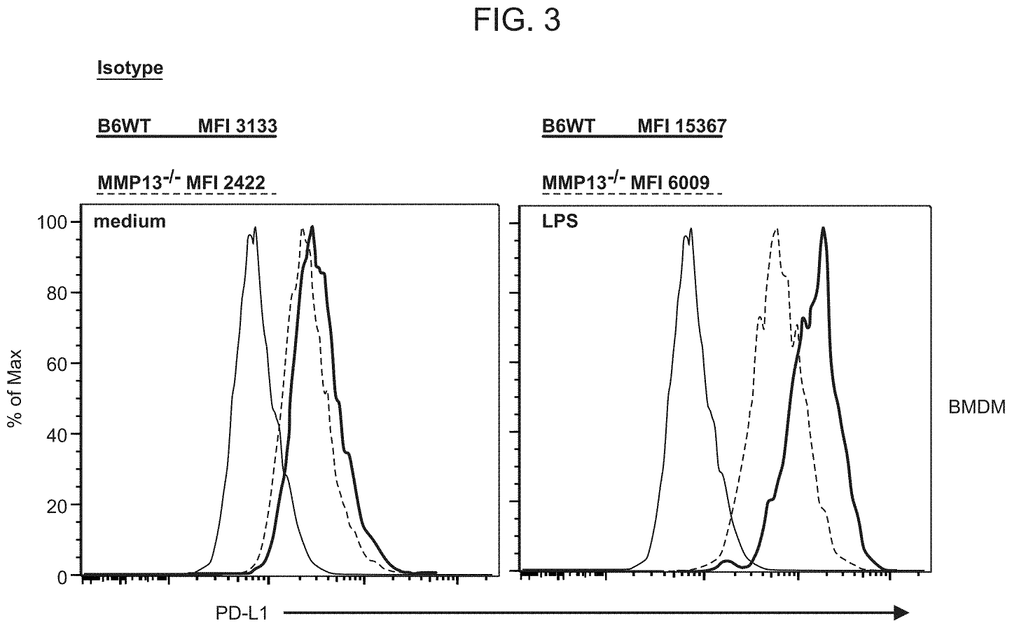

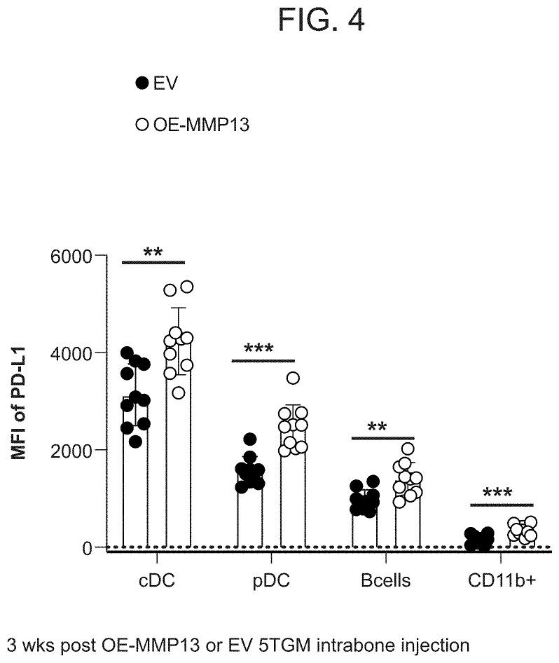

On week 3 following 5TGM1 injection, mice were euthanized, and spleen and tibiae were harvested for flow cytometry analysis. The median fluorescence intensity (MFI) of PD-L1 expression on different APC subpopulation (cDC, pDC, B cells and monocytes) were significantly increased in OE-MMP13 5TGM1 injection group. It suggested that overexpression of MMP13 in 5TGM Myeloma cells promotes expression of PD-L1 in antigen presenting cells of myeloma microenvironment. (FIGS. 3 and 4)

As shown in FIG. 5A, HEK293 cells were transfected with Flag-PD-1H alone or together with c-my tagged full length MMP-13 (FL), E223A enzymatic dead mutant, pro-catalytic domain (aa 1-267) or Hemopexin domain (delta 37-267), followed by IP using anti-c-myc (9E10) antibody. WCL were analyzed as input. As shown in FIG. 5B, HEK293 cells were transfected with MMP-13 alone or together with Flag-PD-1H full length (FL) or extracellular domain (ECD), followed by IP using flag antibody. Whole cell lysates were analyzed as input. As shown in FIG. 5C, purified PD-1H-ECD-Fc recombinant protein was incubated with purified MMP-13 WT-c-myc or E223A-c-myc recombinant protein and pulled down by c-my magnetic beads.

In the description and claims of this specification the word “comprise” and variations of that word, such as “comprises” and “comprising” are not intended to exclude other features, additives, components, integers or steps but rather, unless otherwise stated explicitly, the scope of these words should be construed broadly such that they have an inclusive meaning rather than an exclusive one.

Although the subject peptides and methods of the invention have been described in the present disclosure by way of illustrative examples, it is to be understood that the invention is not limited thereto and that variations can be made as known by those skilled in the art without departing from the teachings of the invention defined by the appended claims.

What is claimed is:

1. A peptide comprising at least six contiguous amino acid residues from the N-terminal domain of MMP-13 [SEQ ID NO: 2], said peptide being capable of inhibiting T cell proliferation.

2. The peptide of claim 1 which comprises at least six contiguous amino acid residues from the pro-peptide region of the N-terminal domain of MMP-13 [SEQ ID NO: 3].

3. The peptide of claim 1 which comprises at least six contiguous amino acid residues from peptides 245-256 of the N-terminal domain of MMP-13 [SEQ ID NO: 4].

4. The peptide of claim 1 which comprises from six to twenty-five contiguous amino acid residues from the N-terminal domain of MMP-13.

5. The peptide of claim 1 which comprises from six to twenty contiguous amino acid residues from the N-terminal domain of MMP-13.

6. The peptide of claim 1 which comprises from six to fifteen contiguous amino acid residues from the N-terminal domain of MMP-13.

7. The peptide of claim 1 selected from TYTGKSHFMLPD (SEQ ID NO: 4), TYTGKS (SEQ ID NO: 5), HFMLPD (SEQ ID NO: 6), TYTGKSHFM (SEQ ID NO: 7), AERYLRS (SEQ ID NO:8), YHPTNLAGIL (SEQ ID NO:9), KENAASSM (SEQ ID NO:10), MTERLREMQ (SEQ ID NO:11), FGLEVTGK (SEQ ID NO:12), LDDNTLDVMKK (SEQ ID NO:13), PRCGVPDVG (SEQ ID NO:14), EYNVFPR (SEQ ID NO:15), KWSKMNLTYR(SEQ ID NO:16), IVNYTPDMT (SEQ ID NO:17), HSEVEKAFKKA (SEQ ID NO: 18), RSYYYHPTNLAGIL (SEQ ID NO:19), YHPTNLAGILKENA (SEQ ID NO:20), and RSYYHPTNLAGILKEN (SEQ ID NO:21).

8. A method of inhibiting T cell proliferation in a subject in need of such inhibition that comprises administering to the subject a T cell proliferation inhibiting amount of the N-terminal domain of MMP-13 [SEQ ID NO: 2] or a peptide comprising at least six contiguous amino acid residues of the N-terminal domain of MMP-13 [SEQ ID NO: 2].

9. The method of claim 8 that comprises administering to the subject a T cell proliferation inhibiting amount of a peptide comprising from six to about twenty-five contiguous amino acid residues of said N-terminal domain of MMP-13.

10. The method of claim 8 wherein the T cell proliferating peptide is selected from TYTGKSHFMLPD (SEQ ID NO: 4), TYTGKS (SEQ ID NO: 5), HFMLPD (SEQ ID NO: 6), TYTGKSHFM (SEQ ID NO: 7), AERYLRS (SEQ ID NO:8), YHPTNLAGIL (SEQ ID NO:9), KENAASSM (SEQ ID NO:10), MTERLREMQ (SEQ ID NO:11), FGLEVTGK (SEQ ID NO:12), LDDNTLDVMKK (SEQ ID NO:13), PRCGVPDVG (SEQ ID NO:14), EYNVFPR (SEQ ID NO:15), KWSKMNLTYR(SEQ ID NO:16), IVNYTPDMT (SEQ ID NO:17), HSEVEKAFKKA (SEQ ID NO: 18), RSYYYHPTNLAGIL (SEQ ID NO:19), YHPTNLAGILKENA (SEQ ID NO:20), and RSYYHPTNLAGILKEN (SEQ ID NO:21).

11. A pharmaceutical composition comprising the N-terminal domain of MMP-13 [SEQ ID NO: 2] or a peptide comprising at least six contiguous amino acid residues of said N-terminal domain of MMP-13 and a pharmaceutically-acceptable carrier.

12. The pharmaceutical composition of claim 11 which comprises a peptide comprising at least six contiguous amino acid residues of the N-terminal domain of MMP-13 [SEQ ID NO: 2] and a pharmaceutically-acceptable carrier.

13. The pharmaceutical composition of claim 12 wherein the composition comprises a peptide comprising from six to about twenty-five contiguous amino acid residues of said N-terminal domain and a pharmaceutically-acceptable carrier.

14. The pharmaceutical composition of claim 10 which comprises a peptide selected from TYTGKSHFMLPD (SEQ ID NO: 4), TYTGKS (SEQ ID NO: 5), HFMLPD (SEQ ID NO: 6), TYTGKSHFM (SEQ ID NO: 7), AERYLRS (SEQ ID NO:8), YHPTNLAGIL (SEQ ID NO:9), KENAASSM (SEQ ID NO:10), MTERLREMQ (SEQ ID NO:11), FGLEVTGK (SEQ ID NO:12), LDDNTLDVMKK (SEQ ID NO:13), PRCGVPDVG (SEQ ID NO:14), EYNVFPR (SEQ ID NO:15), KWSKMNLTYR(SEQ ID NO:16), IVNYTPDMT (SEQ ID NO:17), and HSEVEKAFKKA (SEQ ID NO: 18), RSYYYHPTNLAGIL (SEQ ID NO:19), YHPTNLAGILKENA (SEQ ID NO:20), and RSYYHPTNLAGILKEN (SEQ ID NO:21) and a pharmaceutically-acceptable carrier.


SEQUENCE LISTING (21 sequences)

SEQ ID NO: 1 - protein, 471 aa, Homo sapiens
MHPGVLAAFLFLSWTHCRALPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGPGDEDPNPKHPKTPDKCDPSLSLDAITSLRGETMIFKDRFFWRLHPQQVDAELFLTKSFWPELPNRIDAAYEHPSHDLIFIFRGRKFWALNGYDILEGYPKKISELGLPKEVKKISAAVHFEDTGKTLLFSGNQVWRYDDTNHIMDKDYPRLIEEDFPGIGDKVDAVYEKNGYIYFFNGPIQFEYSIWSNRIVRVMPANSILWC

SEQ ID NO: 2 - protein, 248 aa, Homo sapiens
LPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYG

SEQ ID NO: 3 - protein, 103 aa, Homo sapiens
AERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKA

SEQ ID NO: 4 - protein, 12 aa, synthetic construct
  REGION 1..12: note = Based on Homo sapiens sequence
TYTGKSHFMLPD

SEQ ID NO: 5 - protein, 6 aa, synthetic construct
  REGION 1..6: note = Based on Homo sapiens sequence
TYTGKS

SEQ ID NO: 6 - protein, 6 aa, synthetic construct
  REGION 1..6: note = Based on Homo sapiens sequence
HFMLPD

SEQ ID NO: 7 - protein, 9 aa, synthetic construct
  REGION 1..9: note = Based on Homo sapiens sequence
TYTGKSHFM

SEQ ID NO: 8 - protein, 7 aa, synthetic construct
  REGION 1..7: note = Based on Homo sapiens sequence
AERYLRS

SEQ ID NO: 9 - protein, 10 aa, synthetic construct
  REGION 1..10: note = Based on Homo sapiens sequence
YHPTNLAGIL

SEQ ID NO: 10 - protein, 8 aa, synthetic construct
  REGION 1..8: note = Based on Homo sapiens sequence
KENAASSM

SEQ ID NO: 11 - protein, 9 aa, synthetic construct
  REGION 1..9: note = Based on Homo sapiens sequence
MTERLREMQ

SEQ ID NO: 12 - protein, 8 aa, synthetic construct
  REGION 1..8: note = Based on Homo sapiens sequence
FGLEVTGK

SEQ ID NO: 13 - protein, 11 aa, synthetic construct
  REGION 1..11: note = Based on Homo sapiens sequence
LDDNTLDVMKK

SEQ ID NO: 14 - protein, 9 aa, synthetic construct
  REGION 1..9: note = Based on Homo sapiens sequence
PRCGVPDVG

SEQ ID NO: 15 - protein, 7 aa, synthetic construct
  REGION 1..7: note = Based on Homo sapiens sequence
EYNVFPR

SEQ ID NO: 16 - protein, 10 aa, synthetic construct
  REGION 1..10: note = Based on Homo sapiens sequence
KWSKMNLTYR

SEQ ID NO: 17 - protein, 9 aa, synthetic construct
  REGION 1..9: note = Based on Homo sapiens sequence
IVNYTPDMT

SEQ ID NO: 18 - protein, 11 aa, synthetic construct
  REGION 1..11: note = Based on Homo sapiens sequence
HSEVEKAFKKA

SEQ ID NO: 19 - protein, 14 aa, synthetic construct
  REGION 1..14: note = Based on Homo sapiens sequence
RSYYYHPTNLAGIL

SEQ ID NO: 20 - protein, 14 aa, synthetic construct
  REGION 1..14: note = Based on Homo sapiens sequence
YHPTNLAGILKENA

SEQ ID NO: 21 - protein, 16 aa, synthetic construct
  REGION 1..16: note = Based on Homo sapiens sequence
RSYYHPTNLAGILKEN